(19) United States
(12) Patent Application Publication
(10) Pub. No.: US 2022/0409507 A1
(43) Pub. Date: Dec. 29, 2022

(54) USE OF A GLYCINE BETAINE DERIVATIVE AS AN AGENT FOR CONDITIONING KERATIN FIBRES

(71) Applicant: SURFACTGREEN, COMPIEGNE CEDEX (FR)

(72) Inventors: FRANCIS GALLE, RENNES CEDEX 7 (FR); FREDDY PESSEL, RENNES CEDEX 7 (FR); XAVIER ROUSSEL, LE MANS (FR)

(21) Appl. No.: 17/778,026

(22) PCT Filed: Nov. 6, 2020
(86) PCT No.: PCT/FR2020/052013

§ 371 Date: May 19, 2022

(30) Foreign Application Priority Data
Nov. 22, 2019 (FR) 1913084
Jul. 31, 2020 (FR) 2008156

(51) Int. Cl. A61K 8/42; A61K 8/46; A61K 8/34; A61Q 5/12

(52) U.S. Cl. CPC A61K 8/42; A61K 8/466; A61K 8/342; A61K 8/463; A61Q 5/12

(57) ABSTRACT

The present invention relates to a method for conditioning keratin fibres, comprising topically applying, to the keratin fibres, a cosmetic composition in the form of an emulsion containing, in a cosmetically acceptable medium, a surfactant composition containing at least one ester or amide salt of glycine betaine comprising from 14 to 24 carbon atoms. The invention also relates to the use of a surfactant composition as defined above as an agent for conditioning the keratin fibres.

### SUBJECT OF THE INVENTION

The present invention relates to the use, as a keratin fiber conditioning agent, of a surfactant composition containing at least one glycine betaine ester or amide salt comprising from 14 to 24 carbon atoms. It also relates to a method for conditioning keratin fibers, comprising the topical application, to the keratin fibers, of a cosmetic composition in the form of an emulsion containing, in a cosmetically acceptable medium, a surfactant composition as defined above.

### BACKGROUND OF THE INVENTION

It is well known that hair that has been sensitized, notably damaged and/or embrittled, to various degrees under the action of atmospheric agents or under the action of mechanical and/or chemical treatments, such as dyeing, bleaching and permanent-waving, is often difficult to disentangle, lacks manageability, and the styling thereof and the shaping thereof are particularly difficult. It may also lack sheen, given that its surface may be damaged and may therefore reflect light less uniformly.

To overcome these drawbacks, it is common practice to make use of hair treatments which can condition the hair. These haircare compositions may be conditioning shampoos or hair conditioners, which may be in the form of hair gels or lotions or more or less thick creams containing conditioning agents mainly intended to repair or limit the harmful or undesirable effects brought about by the various treatments or attacking factors to which hair fibers are more or less repeatedly subjected.

Hair treatment compositions have already been proposed that comprise, as conditioning agents, cationic polymers and/or cationic surfactants. These compounds are deposited on the hair and make it possible to improve the state of the fibers and also the cosmetic properties thereof.

However, a certain number of these compounds are not entirely environmentally friendly. There is therefore a need to develop cosmetic compositions which are less ecotoxic, or are not ecotoxic, which make it possible to condition the hair satisfactorily, or even better compared to the compositions of the prior art.

The applicant has now discovered, and expectedly and surprisingly, that certain glycine betaine derivatives can impart advantageous conditioning properties to the hair. These compositions make it possible in particular to improve the disentangling and the smoothing of the hair and also the suppleness thereof, the shaping of the head of hair is easier, and the feel of the hair is very pleasant and fluid. These compounds also make it possible to impart similar properties to other keratin fibers, in particular to the beard.

In the cosmetic field, glycine betaine ester or amide salts or surfactant compositions containing them have already been described for the use thereof in deodorants (WO 2015/003968) or in acid aqueous-based foaming shampoos (WO 2005/121291). Document WO 2015/078890 additionally discloses a surfactant composition obtained by reacting, in a first step, glycine betaine with an alcohol of formula R1-OH containing from 1 to 6 carbon atoms, and then with an alcohol of formula R2-OH having a longer chain, in the presence of a sugar hemiacetal. This two-step process results in a complex surfactant composition containing, besides long-chain glycine betaine ester salts, corresponding esters having a shorter chain, comprising at most six carbon atoms, in a proportion always greater than 15% by weight, and also cationized alkyl polyglycosides. The composition obtained may notably be used in the preparation of haircare products, in particular a disentangling spray.

However, to the knowledge of the applicant, it has never been suggested to use long-chain glycine betaine ester or amide salts to promote the disentangling of the hair, nor proposed to use these compounds in a keratin fiber conditioning product, which is in the form of an emulsion.

### SUMMARY OF THE INVENTION

One subject of the invention is the use, as a keratin fiber conditioning agent, of a surfactant composition containing at least one glycine betaine derivative of formula (1): Xⁿ⁻[(CH₃)₃N⁺—CH₂—COZ—R]ₙ where Z denotes an oxygen atom or an —NH group, R is a saturated or unsaturated, linear or branched alkyl group comprising from 14 to 24 carbon atoms, X is an organic or inorganic anion, and n is equal to 1 or 2.

Another subject of the invention is a method for conditioning keratin fibers, comprising the topical application, to the keratin fibers, of a cosmetic composition in the form of an emulsion containing, in a cosmetically acceptable medium, a surfactant composition as defined above, it being understood that said surfactant composition does not contain alkyl polyglycosides.

### DETAILED DESCRIPTION

The present invention relates to the application, for conditioning keratin fibers, of a surfactant composition based on at least one glycine betaine derivative, which is a long-chain glycine betaine ester or amide salt. These two types of glycine betaine derivatives, and also the methods for preparing same, will now be described in greater detail.

Glycine Betaine Ester Salts

The glycine betaine ester salts may be obtained according to a method comprising the successive steps consisting in:

(1) reacting glycine betaine or a salt thereof with at least one saturated or unsaturated, linear or branched fatty alcohol containing from 14 to 24 carbon atoms, in the presence of an organic or inorganic acid;

(2) cooling the reaction medium to a temperature of from 20° C. to 90° C.; and

(3) collecting the surfactant composition thus obtained.

The first step of this method consists in esterifying the glycine betaine, or trimethylglycine. The glycine betaine may be of plant or synthetic origin. A prior protonation using an organic or inorganic acid, given that glycine betaine is in zwitterionic form (presence of a carboxylate function), is required. The acid may notably be chosen from inorganic acids such as hydrochloric acid, sulfuric acid, perhydrohalic acids, such as perchloric acid, and mixtures thereof. As a variant, it may be chosen from organic acids, such as alkylsulfuric acids, for example decyl or laurylsulfuric acid; arylsulfonic acids, such as benzenesulfonic acid, para-toluenesulfonic acid; alkylsulfonic acids, such as triflic acid, methanesulfonic acid, ethanesulfonic acid, decylsulfonic acid, laurylsulfonic acid or camphorsulfonic acid; sulfosuccinic acid; and mixtures thereof. Lewis acids may also be used. Preferably, it is an alkylsulfonic acid and in particular ethanesulfonic acid, given that it is readily biodegradable, or methanesulfonic acid.

During esterification, the acid function of the salified betaine is reacted with a fatty alcohol, so as to produce a glycine betaine ester in salt form. The term “fatty alcohol” means a saturated or unsaturated, linear or branched (preferably linear) alcohol comprising from 14 to 24 carbon atoms. Examples of such fatty alcohols may be chosen from the group consisting of: myristyl alcohol (C14:0), cetyl alcohol (C16:0), palmitoleyl alcohol (C16:1), stearyl alcohol (C18:0), oleyl alcohol (C18:1), linoleyl alcohol (C18:2), linolenyl alcohol (C18:3), arachidyl alcohol (C20:0), arachidonyl alcohol (C20:4), behenyl alcohol (C22:0), 2-hexyldecanol, 2-octyldodecanol, 2-decyltetradecanol and mixtures thereof. Useable fatty alcohol mixtures may be produced from one or more plant oils and notably from soybean oil, olive oil, sunflower oil, corn oil, palm oil, coconut oil, cottonseed oil, linseed oil, wheat germ oil, safflower oil or rapeseed oil, for example.

It is preferred according to the invention to use one or more alcohols containing from 18 to 22 carbon atoms and more preferentially a mixture of such fatty alcohols.

The esterification reaction generally takes place in the absence of solvent. The water produced during the reaction moreover contributes toward solubilizing the glycine betaine in the reaction mixture.

For the implementation of this reaction, use may for example be made of from 0.8 to 6.0 equivalents, preferably from 0.8 to 2 equivalents, for example from 0.9 to 1.0 equivalent, or as a variant, from 1.1 to 1.8 equivalents, preferentially in this case from 1.2 to 1.6 equivalents and better still from 1.3 to 1.5 equivalents of fatty alcohol or, in a second variant, from 4.0 to 6.0 equivalents, preferentially in this case from 4.5 to 5.5 equivalents and better still from 4.8 to 5.2 equivalents of fatty alcohol.

In addition, advantageously from 1.01 to 3.0 equivalents, preferably from 1.5 to 2.0 equivalents, for example from 1.5 to 1.9 equivalents, and preferentially from 1.5 to 1.7 molar equivalents of organic or inorganic acid or, as a variant (preferably in the second variant above) from 1.02 to 1.08 equivalents, preferentially in this case from 1.03 to 1.07 equivalents and better still from 1.04 to 1.06 molar equivalents of organic or inorganic acid per 1 equivalent of glycine betaine are used. The esterification is carried out at a temperature ranging for example from 120° C. to 180° C., preferably from 150° C. to 180° C. The reaction may be carried out under atmospheric pressure or preferably under reduced pressure, for example at a pressure of from 10 to 600 mbar. Generally, the pressure will be proportionally lower, the greater the chain length of the fatty alcohol involved. The reaction mixture is then cooled to a temperature of from 20° C. to 90° C.

The surfactant composition thus obtained is then collected, said composition containing at least one glycine betaine ester salt of formula Xⁿ⁻[(CH₃)₃N⁺—CH₂—COOR]ₙ where: X is an organic or inorganic anion, R is an alkyl radical corresponding to the R—OH fatty alcohol used in the esterification reaction, and n is equal to 1 or 2.

The X anion is derived from the acid used in the first step of the method and may therefore in particular be a chloride, a sulfate, a perchlorate, an alkylsulfate ion, notably decylsulfate or laurylsulfate, an arylsulfonate ion, notably benzenesulfonate or para-toluenesulfonate, an alkylsulfonate ion, notably triflate, methanesulfonate, ethanesulfonate, decylsulfonate, laurylsulfonate or camphosulfonate, or a sulfosuccinate ion. It is preferred according to the invention for X to be chosen from alkylsulfonates and arylsulfonates, in particular from methanesulfonate, triflate, para-toluenesulfonate and camphorsulfonate ions. It is advantageously the methanesulfonate or ethanesulfonate ion, more preferentially the ethanesulfonate ion.

The R radical may itself be chosen from the following groups: myristyl (C14:0), cetyl (C16:0), palmitoleyl (C16:1), stearyl (C18:0), oleyl (C18:1), linoleyl (C18:2), linolenyl (C18:3), arachidyl (C20:0), arachidonyl (C20:4), behenyl (C22:0), 2-hexyldecyl, 2-octyldodecyl and 2-decyltetradecyl.

It is clearly understood that, in the case where several fatty alcohols are used in the esterification reaction, the surfactant composition obtained according to the invention will comprise several glycine betaine ester salts. The expression “a glycine betaine ester salt” should therefore be understood, in the context of this description and unless otherwise indicated, as referring to one or more of the salts.

The method described above more precisely makes it possible to obtain a surfactant composition containing the following constituents:

(a) at least one glycine betaine ester salt of formula (1): Xⁿ⁻[(CH₃)₃N⁺—CH₂—COO—R]ₙ where R is a saturated or unsaturated, linear or branched alkyl group comprising from 14 to 24 carbon atoms and preferably from 18 to 22 carbon atoms,

(b) at least one fatty alcohol of formula R—OH,

(c) an organic or inorganic acid of formula XH,

(d) a glycine betaine salt of formula Xⁿ⁻[(CH₃)₃N⁺—CH₂—COOH]ₙ, and

(e) optionally at least one dialkyl ether of formula R—O—R, where X is an organic or inorganic anion and n is equal to 1 or 2.

This surfactant composition can be used as it is in the present invention. In this case, it generally contains from 15% to 85% by weight of glycine betaine ester salt.

In a first variant, the surfactant composition contains:

(a) from 65% to 85% by weight, preferably from 70% to 80% by weight, of glycine betaine ester salt,

(b) from 1% to 20% by weight, for example from 1% to 9% by weight or from 10% to 20% by weight, of fatty alcohol,

(c) from 1% to 20% by weight, for example from 5% to 15% by weight, of organic or inorganic acid,

(d) from 1% to 20% by weight, for example from 2% to 15% by weight, of glycine betaine salt,

(e) from 0 to 15% by weight, for example from 2% to 10% by weight, of dialkyl ether.

In a second variant, which is preferred, the surfactant composition contains:

(a) from 15% to 45% by weight, preferably from 20% to 30%, more preferentially from 25% to 30% by weight, of glycine betaine ester salt,

(b) from 55% to 80% by weight, for example from 60% to 65% or from 65% to 70% or from 70% to 80% by weight, of fatty alcohol,

(c) from 0 to 5% by weight, for example from 0 to 1% by weight, of organic or inorganic acid,

(d) from 0 to 3% by weight, for example from 0 to 1% by weight, of glycine betaine salt,

(e) from 0 to 15% by weight, for example from 2% to 10% by weight, of dialkyl ether.

This composition may be obtained by using the method described above, which uses an amount of fatty alcohol of between 4 and 6 equivalents and an amount of acid ranging from 1.02 to 1.08 equivalents, per 1 equivalent of glycine betaine. The composition obtained, which is rich in alcohol and low in acid, has several advantages, compared to the composition according to the first variant, obtained by using from 1.1 to 1.8 equivalents of fatty alcohols and from 1.5 to 2.0 equivalents of acid. Specifically, the presence of a smaller amount of acid in the composition makes it possible to increase its naturalness and to reduce the amount of pH corrector to be added during the formulation of the surfactant composition, this pH corrector itself being capable of having a negative impact on the stability of the emulsion and certain properties imparted to the keratin fibers. The performance of the surfactant composition is also improved by the increase in the amount of residual alcohol that it contains.

Preferentially, the weight ratio of the glycine betaine ester salt to fatty alcohol is between from 20:80 to 30:70.

Advantageously, the surfactant composition does not contain any constituents other than the components (a) to (e) above. As a variant, the above method may include an additional step that consists in isolating the glycine betaine ester salt present in this composition, which may be used as it is in the present invention. In the latter case, the surfactant composition used according to the invention will comprise at least 90%, preferably at least 95%, or at least 99% by weight of glycine betaine derivative.

Glycine Betaine Amide Salts

These glycine betaine derivatives may be prepared according to a process comprising the following successive steps consisting in:

(1) reacting glycine betaine or a salt thereof with a saturated or unsaturated, linear or branched C₄-C₈ alcohol, in the presence of an organic or inorganic acid, at a temperature ranging for example from 100° C. to 180° C. and under reduced pressure;

(2) cooling the reaction medium to a temperature of from 20° C. to 80° C.;

(3) adding one or more alkylamines containing from 14 to 24 carbon atoms;

(4) removing the residual alcohol; and

(5) collecting the surfactant composition thus obtained.

The first step of this method consists of an esterification reaction of the glycine betaine, which may be carried out in a similar manner to the production of the glycine betaine esters, except that use is made of one or more linear and/or branched C₄-C₈ alcohols in the presence of the acid which may be chosen from those described above. Examples of such alcohols include butanol, pentanol, 3-methylbutan-1-ol (or isoamyl alcohol), fusel alcohol (mixture of pentanol, 2-methylbutan-1-ol and 3-methylbutan-1-ol), hexanol, heptanol, octanol and mixtures thereof. The term “butanol” is understood equally in this description to mean n-butanol, isobutanol and sec-butanol. Butanol, and more particularly n-butanol, and also hexanol, are preferred for use in this invention, hexanol being particularly preferred. This reaction is generally carried out in the absence of any solvent, the alcohol used constituting both the reactant and the medium. The water produced during the reaction also contributes to the solubilization of the glycine betaine in the reaction mixture. It is generally possible to use from 1.1 to 20 equivalents, for example from 2 to 4 equivalents, of linear or branched C₄-C₈ alcohol and from 1.0 to 1.5 equivalents of sulfonic acid, for example from 1.0 to 1.2 equivalents and preferentially 1.1 equivalents of sulfonic acid, per 1 equivalent of glycine betaine. The esterification may be carried out at a temperature of from 100° C. to 180° C., preferentially from 100° C. to 160° C., more preferentially from 120° C. to 150° C. or from 130° C. to 160° C. under atmospheric pressure or under reduced pressure.

The product of the esterification reaction may optionally be treated so as to separate the salt of the glycine betaine ester formed from the reaction medium. To do this, it is possible for example to filter the reaction medium, which makes it possible to separate the abovementioned salified ester, which is soluble in alcohol, from the other constituents which are not soluble.

Added next, either to the reaction medium or to the isolated ester, are one or more C14-C24 alkylamine(s). Examples of such amines are: tetradecylamine, hexadecylamine, octadecylamine, docosanylamine, eicosanylamine and mixtures thereof. It is preferred according to the invention to use one or more amines containing from 16 to 22 carbon atoms and more preferentially a mixture of such amines.

In this step, the alkylamine is advantageously used in molten form. The amount of alkylamine(s) added may for example represent from 0.9 to 1.5 equivalents and preferably from 1.0 to 1.2 equivalents per 1 equivalent of glycine betaine initially used. This aminolysis reaction is typically performed at a temperature of from 50° C. to 180° C. and preferably from 120° C. to 140° C., under reduced pression, for example under a pressure of from 1 to 30 mbar. At the same time as the aminolysis reaction, the alcohol is removed by distillation under reduced pressure.

The aminolysis reaction and the distillation are carried out for a time of from 1 to 7 hours, notably from 3 to 5 hours.

The surfactant composition thus obtained is then collected.

This method makes it possible to obtain a surfactant composition comprising:

(a) one or more glycine betaine amide salts of formula (1): Xⁿ⁻[(CH₃)₃N⁺—CH₂—CONH—R]ₙ where R is a saturated or unsaturated, linear or branched alkyl group comprising from 14 to 24 carbon atoms and preferably from 16 to 22 carbon atoms;

(b) one or more alkylammonium salts of formula (2): Xⁿ⁻[NH₃⁺R]ₙ where R is a saturated or unsaturated, linear or branched alkyl group comprising from 14 to 24 carbon atoms and preferably from 16 to 22 carbon atoms;

(c) one or more glycine betaine ester salts of formula (3): Xⁿ⁻[(CH₃)₃ N⁺—CH₂—COOR′]ₙ where R′ is a saturated or unsaturated, linear or branched alkyl radical containing from 4 to 8 carbon atoms; and

(d) glycine betaine of formula (4): (CH₃)₃N⁺—CH₂—COO⁻ where X is an organic or inorganic anion and n is equal to 1 or 2.

This surfactant composition may be used as it is in the present invention. In this case, it generally contains from 60% to 98% by weight, for example from 70% to 80% by weight, of glycine betaine amide salt. The constituent (b) may represent from 0 to 25% by weight, for example from 15% to 20% by weight, the constituent (c) from 0 to 15% by weight, for example from 5% to 10% by weight, and the constituent (d) from 0 to 5% by weight, relative to the total weight of the surfactant composition. Advantageously, this surfactant composition does not contain any constituents other than the components (a) to (d) above. As a variant, the above method may include an additional step that consists in isolating the glycine betaine amide salt present in this composition, which may be used as it is in the present invention. In the latter case, the surfactant composition used according to the invention will comprise at least 90%, preferably at least 95%, or at least 99% by weight of glycine betaine derivative.

It is preferred in any case for the surfactant composition containing a glycine betaine ester or amide salt as defined above to not contain cationized or non-cationized alkyl polyglycosides and/or, in the case of the esters (Z═O), for it further to be free of glycine betaine derivative of formula (1′) Xⁿ⁻[(CH₃)₃N⁺—CH₂—COO—R]ₙ where R e is a saturated or unsaturated, linear alkyl group comprising from 1 to 6 carbon atoms, X is an organic or inorganic anion, and n is equal to 1 or 2.

In one preferred embodiment, the surfactant composition according to the invention contains at least 90% by weight, preferably at least 95% by weight, or at least 99% by weight, of ingredients of natural origin, as calculated according to the ISO-16128 standard.

Cosmetic Compositions

For the implementation of the present invention, use is made of a cosmetic composition in the form of an emulsion containing a surfactant composition as described above. This emulsion may have a liquid or semi-liquid consistency, a soft consistency of cream or balm type or a solid consistency of stick type. It may be of oil-in-water (O/W), oil-in-glycerol, water-in-oil (W/O), water-in-glycerol or multiple (for example W/O/W) type. This emulsion is preferentially of oil-in-water type. It generally contains from 1% to 8% by weight, and preferentially from 1% to 4% by weight, of glycine betaine derivative used according to the invention.

This cosmetic composition may notably be packaged in a tube, a pump-dispenser bottle or a jar. As a variant, it may be packaged in an aerosol container, in order to ensure an application of the composition in vaporized form. In the latter case, the cosmetic composition preferably comprises at least one propellant.

The cosmetic composition used according to the invention comprises a cosmetically acceptable medium, i.e. a medium that is compatible with the keratin fibers and the skin, in particular with the hair and the scalp, and that does not give rise to irritation of the skin or scalp or other undesirable effect after application on the keratin fibers.

This cosmetic composition comprises an aqueous phase comprising water, one or more cosmetically acceptable water-soluble solvents chosen from C1-C4 alcohols, such as ethanol, isopropanol, tert-butanol or n-butanol, polyols such as glycerol, propylene glycol and polyethylene glycols, and mixtures thereof. As a variant, it may comprise a mixture of water and one or more of the abovementioned solvents. Preferably, the cosmetic composition has a total water content of between 5% and 95% by weight, preferably between 10% and 90%, for example between 40% and 85% by weight, notably between 50% and 80% by weight relative to the total weight of the composition. The pH of this composition generally varies from 3 to 9, preferably from 3 to 7, preferentially from 3.5 to 6, and better still from 3.5 to 5. It may be adjusted within this range using at least one pH adjuster, chosen for example from: sodium or calcium gluconate, sodium lactate, sodium glycinate, sodium citrate and lactic acid/sodium lactate, acetic acid/sodium acetate and gluconic acid/sodium gluconate buffer solutions.

It further comprises at least one fatty phase containing at least one fatty substance, so as to form an emulsion. Preferably, the fatty substance(s) is (are) chosen from oils, pasty fatty substances, waxes and mixtures thereof. The term “oils” is understood to mean a compound that is liquid at room temperature (25° C.) and atmospheric pressure (10⁵ Pa) which, when it is introduced in a proportion of at least 1% by weight into water at 25° C., is not soluble at all in water, or is soluble up to less than 10% by weight, relative to the weight of oil introduced into the water. The term “pasty fatty substances” is understood to mean fatty substances with a reversible solid/liquid change of state, having an anisotropic crystalline organization in the solid state, and comprising, at a temperature of 23° C., a liquid fraction and a solid fraction, such as plant butters. The term “wax” denotes, in the context of this description, a fatty substance that is solid at 25° C., with a reversible solid/liquid change of state, having a melting point generally of between 30° C. and 160° C., preferably between 50° C. and 90° C., as measured by differential scanning calorimetry (DSC).

Preferably, the cosmetic composition used according to the invention comprises at least one oil. As examples of oils, mention may notably be made of fatty alcohols, fatty esters, hydrocarbons of plant or mineral origin, triglycerides and the plant oils containing them, and mixtures thereof. As fatty alcohols, mention may notably be made of branched and/or unsaturated C10-C20 fatty alcohols such as octyldodecanol and oleyl alcohol. Examples of fatty esters are esters of acids and of monoalcohols chosen from: monoesters and polyesters of C2-C10 (preferably C6-C10) saturated linear acids and of C10-C18 (preferably C10-C14) saturated linear monoalcohols, monoesters and polyesters of C10-C20 saturated linear acids and of C3-C20 (preferably C3-C10) branched or unsaturated monoalcohols; monoesters and polyesters of C5-C20 branched or unsaturated acids and of C5-C20 branched or unsaturated monoalcohols; monoesters and polyesters of C5-C20 branched or unsaturated acids and of C2-C4 linear monoalcohols. Examples of such fatty esters are notably the mixture of coco caprate and caprylate, ethyl macadamiate, shea butter ethyl ester, isostearyl isostearate, isononyl isononanoate, ethylhexyl isononanoate, hexyl neopentanoate, ethylhexyl neopentanoate, isostearyl neopentanoate, isodecyl neopentanoate, isopropyl myristate, octyldodecyl myristate, isopropyl palmitate, ethylhexyl palmitate, hexyl laurate, isoamyl laurate, cetostearyl nonanoate, propylheptyl caprylate, diisopropyl adipate, diethylhexyl adipate, diisopropyl sebacate and diisoamyl sebacate.

As hydrocarbons, mention may be made of squalane (C30), notably plant squalane extracted from olive oil or by biosynthesis, and hemisqualane (C15). Examples of triglycerides are triglycerides of C6-C12 fatty acids, such as triglycerides of caprylic and capric acids and triheptanoin. Examples of plant oils are notably wheatgerm oil, sunflower oil, argan oil, hibiscus oil, coriander oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, blackcurrant oil, evening primrose oil, lavender oil, borage oil, millet oil, barley oil, quinoa oil, rye oil, safflower oil, candlenut oil, passion flower oil, musk rose oil, echium oil, camelina oil or camellia oil.

The fatty substances may represent from 1% to 30% by weight, preferably from 5% to 25% by weight, and preferentially from 10% to 20% by weight, relative to the total weight of the cosmetic composition.

The cosmetic composition used according to the invention may also comprise at least one standard cosmetic ingredient, notably chosen from nonionic surfactants, cationic surfactants, anionic surfactants, amphoteric surfactants; sunscreens, active agents such as vitamins, antidandruff agents, antiseborrheic agents, agents for preventing hair loss and/or for promoting hair regrowth; antioxidants; nacreous agents and/or opacifiers; pigments; fillers; sequestrants; thickeners; non-thickening polymers such as amino silicones and/or cationic polymers; fragrances; preservatives; and mixtures thereof.

The organic acids capable of being used in this composition have a pKa of less than or equal to 7, preferably less than or equal to 6, ranging in particular from 1 to 6, preferably from 2 to 5. According to a preferred embodiment, the organic acid(s) are chosen from carboxylic acids, sulfonic acids and mixtures thereof. In particular, the organic acid(s) are chosen from α- and β-hydroxy acids such as lactic acid, citric acid, glycolic acid, salicylic acid, malic acid, tartaric acid and mixtures thereof, and more preferentially citric acid or lactic acid.

Preferably, the anionic surfactants are chosen from alkylcarbonylisethionic acid salts, such as those identified under the INCI names SODIUM COCOYL ISETHIONATE and SODIUM COCOYL METHYL ISETHIONATE; lactylic acid salts, such as SODIUM LAUROYL LACTYLATE; N-acyl amino acid salts, such as SODIUM LAUROYL GLYCINATE, SODIUM LAUROYL SARCOSINATE, SODIUM LAUROYL TAURATE and SODIUM OLIVOYL GLUTAMATE; anionic sulfate surfactants, notably chosen from alkyl sulfate salts, notably SODIUM COCO SULFATE and POTASSIUM LAURYL SULFATE, C8-C14 alkyl ether sulfate alkyl salts such as SODIUM LAURYL ETHER SULFATE; soaps in the form of carboxylic acid salts, notably SODIUM OLIVATE and SODIUM PALMITATE; and alkyl ether carboxylic surfactants, such as lauryl ether carboxylic acids or sodium lauryl ether carboxylates.

The nonionic surfactant(s) used in the cosmetic composition are preferentially chosen from: saturated or unsaturated, linear or branched, oxyethylenated C8 to C40 alcohols comprising from 1 to 100 mol of ethylene oxide, preferably from 2 to 50 and more particularly from 2 to 40 mol of ethylene oxide and preferably comprising one or two fatty chains; saturated or unsaturated oxyethylenated plant oils comprising from 1 to 100 and preferably from 2 to 50 mol of ethylene oxide; (C8-C30)alkyl (poly)glucosides, which are optionally oxyalkylenated (0 to 10 EO) and comprise from 1 to 15 glucose units; sucrose esters such as sucrose stearate and sucrose distearate, monoglycerolated or polyglycerolated C8 to C40 alcohols, comprising from 1 to 50 mol of glycerol and preferably from 1 to 10 mol of glycerol; saturated or unsaturated, linear or branched, oxyalkylenated C8 to C30 fatty acid amides; esters of saturated or unsaturated, linear or branched, C8 to C30 acids and of polyethylene glycols; preferably oxyethylenated esters of saturated or unsaturated, linear or branched, C8 to C30 acids and of sorbitol; and mixtures thereof.

The amphoteric surfactant(s), preferably non-silicone surfactants, used in the cosmetic composition used in the present invention may notably be optionally quaternized secondary or tertiary aliphatic amine derivatives, in which the aliphatic group is a linear or branched chain containing from 8 to 22 carbon atoms, said amine derivatives containing at least one anionic group, for instance a carboxylate, sulfonate, sulfate, phosphate or phosphonate group. Mention may in particular be made of (C8-C20)alkylbetaines, (C8-C20)alkylsulfobetaines, (C8-C20)alkylamido(C3-C8)alkylbetaines and (C8-C20)alkylamido(C6-C8)alkylsulfobetaines.

The cationic surfactant(s) optionally used in addition to the glycine betaine derivatives may be chosen from optionally polyoxyalkylenated primary, secondary or tertiary fatty amine salts, quaternary ammonium salts, and mixtures thereof.

The thickener(s) may be chosen from cellulose thickeners, for example hydroxyethylcellulose, hydroxypropylcellulose and carboxymethylcellulose; gums of natural origin such as tara gum (Caesalpinia spinosa gum) and guar gum and its derivatives, for example hydroxypropyl guar and guar hydroxypropyltrimonium chloride; gums of microbial origin, such as xanthan gum and scleroglucan gum; synthetic thickeners such as crosslinked homopolymers of acrylic acid or of acrylamidopropanesulfonic acid; or nonionic, anionic, cationic or amphoteric associative polymers. Among the cationic polymers that can be used as thickening polymers, mention may more particularly be made of polymers of polyamine, poly(aminoamide) and poly(quaternary ammonium) type, notably cationic celluloses, cationic guar gums and homopolymers or copolymers of dimethyldiallylammonium halides.

Examples of active agents that may be included in the composition according to the invention are sodium hyaluronate, tocopherol and its derivatives such as tocopherol acetate, panthenol, serine, glycerol, arginine, ceramides such as 2-oleamido-1,3-octadecanediol, hydroxypropyl starch phosphate and mixtures thereof without this list being limited. Mention may also be made of hair conditioning agents such as silicones, in particular dimethicone and amodimethicone.

Method/Use

The cosmetic composition used according to the invention is in the form of a care product for keratin fibers, in particular a conditioner or hair mask intended for treating the hair. In particular, it is intended for treating, and therefore is preferably applied to, weakened and/or damaged hair, for example hair weakened and/or damaged by chemical or mechanical treatments, notably by dyeing, bleaching, permanent waving or straightening or by brushing. It may also be used as treatment cream shampoo, notably antiseborrheic or antidandruff shampoo. This composition may constitute a rinse or no-rinse product. It does not generally have foaming properties. This composition may also be in the form of a rinse-out product, to be applied before or after dyeing, bleaching, permanent waving or straightening or else between the two steps of a permanent waving or straightening operation.

As a variant, the cosmetic composition according to the invention may be in the form of a beard care product.

The present invention relates more specifically to a cosmetic method for conditioning keratin fibers, comprising the topical application, to the keratin fibers, of a cosmetic composition in the form of an emulsion as described above. The term “keratin fibers” is understood to mean head hair and body hair, notably the beard and the eyebrows. The types of hair to which the composition according to the invention may be applied include Caucasian, African and Asian. They may be more or less curly or even kinky. The term “conditioning” is understood within the context of this description to mean the improvement in at least one property of the keratin fibers chosen from: their combability, their disentanglability, their softness, their suppleness, their sheen and their manageability. Preferably, the conditioning of the keratin fibers does not include the cleansing thereof. The composition according to the invention does not therefore generally constitute a shampoo.

The composition may be applied to dry or wet hair, and preferably to wet or damp hair, i.e. that has been prewashed and rinsed. According to one embodiment, the method according to the invention consists in applying an effective amount of the cosmetic composition to the hair, optionally kneading the hair, optionally leaving the composition on the hair, and rinsing. The leave-in time of the composition on the hair may be between a few seconds and 15 minutes and preferably between 30 seconds and five minutes. The composition is generally rinsed with water. An optional step of drying the hair may be carried out. In another embodiment, the method according to the invention consists in applying an effective amount of the cosmetic composition to the hair, optionally kneading the fibers, optionally leaving the composition on said fibers, and optionally drying without prior rinsing.

This method is more particularly intended to improve the combability and/or the softness and/or the suppleness and/or the manageability and/or the sheen of the keratin fibers and/or to smooth them and/or to hydrate them and/or to reduce the static electricity thereof. It is not generally suitable or intended for cleansing the keratin fibers.

### FIGURES

FIG. 1 illustrates the combability of a lock of hair treated respectively with water and with compositions containing a reference surfactant, a mixture of glycine betaine ester salts according to the invention and a mixture of glycine betaine amide salts according to the invention, respectively.

FIG. 2 is a chart illustrating the softness of locks treated using compositions containing a reference surfactant, a mixture of glycine betaine ester salts according to the invention and a mixture of glycine betaine amide salts according to the invention, respectively, in comparison with a commercial hair detangler.

### EXAMPLES

The invention will be better understood in light of the following examples, which are given for purely illustrative purposes and are not intended to limit the scope of the invention, defined by the appended claims.

### Example 1: Synthesis of Surfactant Compositions Based on Glycine Betaine Ester Salts

Synthesis of Methanesulfonic Acid Salts

Glycine betaine (1.0 eq) and a mixture of C18 to C22 fatty alcohols (1.4 eq) are introduced into a reactor. The setpoint temperature in the mixture is set at 170° C. and the pressure is reduced to a value of 60 mbar. Once the pressure and temperature setpoints are reached, a 70% methanesulfonic acid solution (1.6 eq) is added to the reaction mixture. As soon as the addition is completed, the setpoint temperature is brought to 150° C. and the pressure is maintained at a value of 30 mbar. Five hours after the start of the introduction of the acid, the reaction mixture is cooled to 80° C., then the product is recovered, cooled to room temperature, and constitutes the surfactant composition according to the invention, which contains the following constituents:

TABLE 1

| | Composition by weight |
|---|---|
| C18-C22 alkyl betainate mesylate | 66.3% |
| Glycine betaine mesylate | 3.4% |
| C18-C22 fatty alcohols | 14.2% |
| Methanesulfonic acid | 7.4% |
| C18-C22 alkyl ethers | 8.7% |

Synthesis of Ethanesulfonic Acid Salts

Glycine betaine (1.0 eq) and a mixture of C18 to C22 fatty alcohols (5.0 eq) are introduced into a reactor. The setpoint temperature in the mixture is set at 170° C. and the pressure is reduced to a value of 60 mbar. Once the pressure and temperature setpoints are reached, a 70% ethanesulfonic acid solution (1.05 eq) is added to the reaction mixture. As soon as the addition is completed, the setpoint temperature is brought to 150° C. and the pressure is maintained at a value of 30 mbar. Six hours after the start of the introduction of the acid, the reaction mixture is cooled to 80° C., then the product is recovered, cooled to room temperature, and constitutes the surfactant composition according to the invention, which contains the following constituents:

TABLE 2

| | Composition by weight |
|---|---|
| C18-C22 alkyl betainate esylate | 27.7% |
| Glycine betaine esylate | 0.4% |
| C18-C22 fatty alcohols | 66.7% |
| Ethanesulfonic acid | 0.6% |
| C18-C22 alkyl ethers | 4.5% |

### Example 2: Synthesis of Surfactant Compositions Based on Glycine Betaine Amide Salts

Synthesis of Methanesulfonic Acid Salts

Glycine betaine (1.0 eq), butanol (3.0 eq) and a 70% methanesulfonic acid solution (1.1 eq) are introduced into a reactor on which a condenser is mounted. The mixture is heated to 140° C. at atmospheric pressure. After 3 hours of reaction, a Dean-Stark trap filled with butanol is mounted on the reactor. The mixture is left at atmospheric pressure since the distillation of the water-butanol azeotrope is sufficiently pronounced at the start. After a further 3 hours of reaction, when the distillation rate of the water-butanol azeotrope has decreased, the pressure is reduced to 700 mbar in order to accelerate the removal of the water and to enable the equilibrium to be shifted towards the glycine betaine butyl ester. The degree of conversion is monitored by ¹H NMR analyses.

The NMR method consists in acquiring a ¹H spectrum of the sample dissolved in a CDCl₃/CD₃OD mixture (1/1, v/v), taking the methanol signal at 3.31 ppm as reference. The characteristic signals of the various compounds are then integrated: MsOGBOBu (4.35 ppm, s, 2H), MsOGB (4.28 ppm, s, 2H), butanol (3.53 ppm, t, 2H), methanesulfonate (2.74 ppm, s, 3H), dibutyl ether (3.40 ppm, t, 4H), where XOGBOBu denotes the glycine betaine ester sulfonate salt formed and XOGB denotes the glycine betaine sulfonate formed. The characteristic signal of the methanesulfonate takes into account both the methanesulfonic acid present in the medium, and also the methanesulfonate which is the counterion of glycine betaine and of butyl betainate mesylate (MsOGBOBu).

The degree of conversion of the reaction is obtained by means of the integration values via the following calculation:

$$η = \frac{I \text{?} / 2}{I \text{?} / 2 + I \text{?} / 2} = \frac{I \text{?}}{I \text{?} + I \text{?}}$$

$$\text{?} \text{indicates text missing or illegible when filed}$$

where:

[symbol] is the degree of conversion

Iᵢ is the integration value of the characteristic signal of the compound i.

Once the degree of conversion of the esterification reaction reaches 96%, the reaction mixture is allowed to cool to 60° C. During this cooling phase, the Dean-Stark assembly is replaced with distillation apparatus and the reactor is placed under reduced pressure so as to remove a portion of the butanol and the remaining traces of water in the reaction mixture. Once the mixture is at 60° C., the mixture of C16-C22 fatty amines (1.1 eq) which have been melted beforehand is added. The reaction mixture is then heated to 150° C. under reduced pressure. The pressure is gradually reduced to 10 mbar. After total distillation of the butanol (about 4 hours), the reaction mixture is recovered and constitutes the surfactant composition according to the invention, containing the following constituents:

TABLE 3

| | Composition by weight |
|---|---|
| Betainylamino(C16-C22)alkane | 73.1% |
| mesylate | |
| (C16-C22)Alkylammonium mesylate | 17.7% |
| Butyl mesylate betainate | 8.0% |
| Glycine betaine | 1.2% |
| Butanol | 0.0% |

Synthesis of Ethanesulfonic Acid Salts

Glycine betaine (1.0 eq) and hexanol (3.0 eq) are introduced into a reactor on which a Dean-Stark trap filled with hexanol is mounted. Fixed to the cover of the reactor is an isobaric dropping funnel containing a 70% ethanesulfonic acid solution (1.1 eq). The mixture is stirred and heated to 150° C. under pressure reduced to 600 mbar. Once the reaction conditions are reached, the 70% ethanesulfonic acid solution is gradually introduced into the reaction mixture. Once the addition is completed, the pressure is steadily reduced until it reaches 400 mbar in order to accelerate the removal of the water and to enable the equilibrium to be shifted towards the glycine betaine ester. The degree of conversion is monitored by ¹H NMR analyses.

The NMR method consists in acquiring a ¹H spectrum of the sample dissolved in a CDCl₃/CD₃OD mixture (1/1, v/v), taking the methanol signal at 3.31 ppm as reference. The characteristic signals of the various compounds are then integrated: EsOGBOC6 (4.35 ppm, s, 2H), EsOGB (4.28 ppm, s, 2H), hexanol (3.53 ppm, t, 2H), ethanesulfonate (2.82 ppm, q, 3H), dihexyl ether (3.40 ppm, t, 4H), where XOGBOC6 denotes the glycine betaine ester sulfonate salt formed and XOGB denotes the glycine betaine sulfonate formed. The characteristic signal of the ethanesulfonate takes into account both the ethanesulfonic acid present in the medium, and also the ethanesulfonate which is the counterion of glycine betaine and of hexyl betainate esylate (EsOGBOC6).

The degree of conversion of the reaction is obtained by means of the integration values via the following calculation:

$$η = \frac{I \text{?} / 2}{I \text{?} / 2 + I \text{?} / 2} = \frac{I \text{?}}{I \text{?} + I \text{?}}$$

$$\text{?} \text{indicates text missing or illegible when filed}$$

where:

[symbol] is the degree of conversion

Iᵢ is the integration value of the characteristic signal of the compound i.

Once the degree of conversion of the esterification reaction reaches 96%, the reaction mixture is allowed to cool to 80° C. During this cooling phase, the Dean-Stark assembly is replaced with distillation apparatus and the reactor is placed under reduced pressure so as to remove a portion of the hexanol and the remaining traces of water in the reaction mixture. Once the mixture is at 80° C., a mixture of C16-C22 fatty amines (1.1 eq) which have been melted beforehand is added. The reaction mixture is then heated to 150° C. under reduced pressure. The pressure is gradually reduced to 5 mbar. After total distillation of the hexanol (about 4 hours), the reaction mixture is recovered and constitutes the surfactant composition according to the invention, which contains the following constituents:

TABLE 4

| | Composition by weight |
|---|---|
| Betainylamino(C16-C22)alkane esylate | 71.4% |
| (C16-C22) Alkylammonium esylate | 18.9% |
| Hexyl esylate betainate | 8.8% |
| Glycine betaine | 1.0% |
| Hexanol | 0.0% |
| Dihexyl ether | 0.0% |

### Example 3: Disentangling Test (Sensory Test)

A comparative test was carried out for disentangling a lock of hair using a fatty alcohol-in-water emulsion containing, as conditioning agent, either a mixture of C18 to C22 glycine betaine ester salts or a mixture of C16 to C22 glycine betaine amide salts according to the invention, or a conditioning agent, namely behentrimonium chloride (Varisoft® BT 85 from EVONIK).

The glycine betaine ester and amide salts corresponded respectively to the compositions presented in table 1 of example 1 and in table 3 of example 2.

These emulsions had the following composition:

| | |
|---|---|
| Cetyl alcohol | 6.00%-10.00%* |
| Conditioning agent (as active material) | 3.00% |
| Preservative | 0.60% |
| Buffer solution | qs pH 4.0-5.0 |
| Demineralized water | QS for 100.00% |

*So as to achieve a viscosity of from 7000 to 22,000 mPa · s (LV4, 20 rpm, 20° C.)

All these emulsions had the appearance of an opaque viscous cream.

Tap water (hardness 30° F., temperature: 37° C.) was furthermore used as control.

To carry out this test, 2 locks of hair were dampened beforehand then wrung out and finally rubbed 15 times in the palm of the hand to entangle the hair. 1 g of each product was then applied to one of the two damp locks which was then massaged 8 times over its entire length to distribute the product properly. After a leave-on time of 3 minutes, the locks were rinsed with tap water then wrung out by hand. After having laid them on a flat surface, the number of comb strokes necessary to obtain a lock which could be combed without constraint was measured. This procedure was repeated three times per product, on 3 different locks. Only one test was carried out with tap water (30° F.).

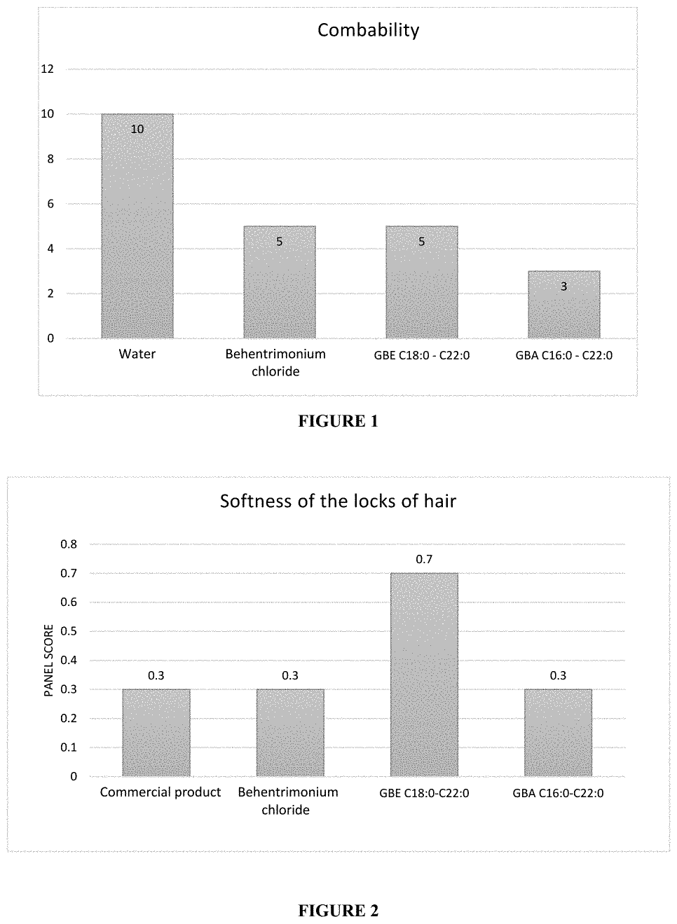

The results of these tests are illustrated in FIG. 1. As revealed in this figure, the glycine betaine ester salts according to the invention offer a performance equivalent to that of the reference surfactants. However, the glycine betaine derivatives have a greater biodegradability than the reference surfactants, are 100% bio-based and their method of synthesis is more environmentally friendly. The glycine betaine amide salts have a greater efficacy than the ester salts.

Sensory analysis of the softness of the locks thus obtained was then carried out by a trained panel, in comparison with a lock treated in the same way as above, but with a commercial disentangling product (Elsève® Total Repair Rapid Restore) containing the same content of cationic conditioning agent (behentrimonium chloride) and having the same pH and substantially the same viscosity as the emulsions above.

The results of this evaluation are presented in FIG. 2. As shown in this figure, the locks treated with the cosmetic composition according to the invention containing glycine betaine ester salts are perceived to be much softer than those treated with the composition containing the reference surfactants and even with the commercial disentangling agent.

An additional test was carried out under the same conditions, using the surfactant composition presented in table 2 of example 1. All of the results are assembled in the table below:

TABLE 5

| | Mean number of comb | |
|---|---|---|
| Product tested | passes | Mean softness |
| GBE Table 1 - Ex 1 | 5.0 | 0.7 |
| GBE Table 2 - Ex 1 | 5.7 | 2.0 |
| GBA Table 3 - Ex 2 | 3.0 | 0.3 |
| Control product | 5.0 | 0.3 |

It is observed that the performance of this surfactant composition is much better than that of a surfactant composition that is similar but that is prepared in the presence of a smaller amount of fatty alcohol and a greater amount of acid.

### Example 4: Disentangling Test (Mechanical Test)

Materials and Method

Use is made of a Diastron® Fibra One machine equipped with a comb in order to measure the work (in Joules) needed to travel through a lock of hair.

To do this, five calibrated flat locks (3.5 g; 28 cm) of bleached Caucasian hair are firstly washed using 1 ml of sodium lauryl ether sulfate solution (28% active material). The locks are rubbed 20 times between the hands, then rinsed for 1 min 30 sec in water. This washing is then repeated twice, and then the excess water is removed by wringing out the lock 3 times between 2 fingers. The machine is regulated in the following:

Starting position: 75 mm

Combing length: 200 mm

Speed: 2000 mm/min

Three measurements are carried out for each lock, namely one measurement after each rinsing step, then the mean of the three measurements is calculated.

The same conditioner treatment (0.5 ml) is then applied on one side of each of the five locks, then the product is spread 10 times with two fingers before being applied (0.5 ml) to the other side of the lock and then spread 10 times with two fingers. The locks are then each rinsed for 16 seconds with tap water (changing side every 8 sec). The excess water is removed by wringing out 3 times between 2 fingers with the same force. The locks are then again tested on the Diastron® as described above. Next, they are subjected to two successive rinsings (passing under tap water for 10 seconds then removal of the excess water by wringing out 3 times between 2 fingers) and are again passed to the Diastron® after each rinsing. The mean of the three measurements obtained is calculated.

For each lock, the percentage decrease in force needed for the disentangling of the lock is then determined using the following formula: D=(W_{T}−W_{O})/100, where W_{T} is the work measured after treatment and W_{O} is the work measured before treatment. The mean DM of the decrease percentages obtained for the five locks is then calculated.

The conditioner products tested were the following:

1—Surfactant composition according to example 2, containing approximately 1% by weight of glycine betaine ester and 3% by weight of C18-22 alcohols,

2—Comparative surfactant composition, containing 1% by weight of nonionic surfactant (sorbitan stearate) and 3% by weight of C18-22 alcohols,

3—Comparative surfactant composition, containing 1% by weight of behentrimonium chloride as cationic surfactant and 3% by weight of C18-22 alcohols,

4—Comparative surfactant composition, containing 1% by weight of cetrimonium chloride as cationic surfactant and 3% by weight of C18-22 alcohols,

where “C18-22 alcohols” denotes a mixture of fatty alcohols containing from 18 to 22 carbon atoms (Stenol® 1822A from BASF).

Results

The results of the tests described above are assembled in table 6 below.

TABLE 6

| | | Standard |
|---|---|---|
| Conditioner product | DM (%) | deviation |
| 1 | 91.1 | 0.4 |
| 2 | 23.5 | 12.7 |
| 3 | 91.4 | 2.4 |
| 4 | 83.6 | 3.2 |

As it emerges from this table, the product according to the invention (product 1) gives the treated locks an ease of disentangling, which is expressed by the reduction in the work measured for combing the locks. This reduction is of the same order as that obtained with a non-biodegradable cationic surfactant which is customarily used in disentangling products (product 3) and higher than that obtained with a commercial cationic surfactant having a lower biodegradability than the product according to the invention (product 4). The performance of the product according to the invention is moreover much better than that obtained with a nonionic surfactant (product 2).

### Example 5: Formulations

Several types of products can be prepared using surfactant compositions according to the invention, based respectively on palmityl (GBE C16:0 or GBA C16:0), stearyl (GBE C18:0 or GBA C18:0), arachidyl (GBE C20:0 or GBA C20:0), or behenyl (GBE C22:0 or GBA C22:0) ester or amide salts or mixtures thereof, and more particularly esters according to the second variant of the invention, containing at least 55% by weight of fatty alcohol.

Examples of such products are indicated below, the ingredients in capitals being identified by their INCI names.

Conditioner:

| Ingredients | % Material |
|---|---|
| GBE C16:0/C18:0 | 12% |
| Lactic acid/sodium lactate | qs for pH 4.0 |
| buffer solution | |
| Fragrance | 0.25% |
| Colorant | 0.02% |
| Preservative | 0.01% |
| Demineralized water | QS for 100% |

Hair Mask:

| Ingredients | % Material |
|---|---|
| GBEC18:0/C22:0 | 12% |
| Olive oil | 5.00% |
| Gluconic acid/sodium | qs pH 4.0 |
| gluconate buffer solution | |
| Panthenol | 0.15% |
| Fragrance | 0.05% |
| Colorant | 0.02% |
| Preservative | 0.01% |
| Demineralized water | qs for 100% |

2 in 1 Solid Shampoo:

| Ingredients | % Material |
|---|---|
| SODIUM COCOYL | 35% |
| ISETHIONATE | |
| STEARIC ACID (AND) | 15% |
| PALMITIC ACID | |
| SODIUM OLIVOYL | 10% |
| GLUTAMATE | |
| GBAC16:0/C22:0 | 15% |
| GLYCERIN | 8% |
| CORYLUS AVELLANA | 5% |
| SEED OIL | |
| Cornstarch | 4% |
| SUCROSE STEARATE | |
| GLYCERYL STEARATE | 4% |

2 in 1 Liquid Shampoo:

| Ingredients | % Material |
|---|---|
| SODIUM LAURYL SULFATE | 14% |
| COCAMIDOPROPYL | 7% |
| BETAINE | |
| GBEC18:0/C22:0 | 17% |
| GLYCERIN | 4% |
| POLYSORBATE 80 | 2% |
| SUCROSE PALMITATE | 2.5% |
| GLYCERYL BEHENATE | 1.5 |
| CAESALPINIA SPINOSA | 1% |
| GUM | |
| PHENOXYETHANOL (AND) | 1% |
| CAPRYLYL GLYCOL | |
| Colorant | 0.30% |
| Demineralized water | qs for 100% |

Hair Smoothing Product:

| Ingredients | % Material |
|---|---|
| GBEC18:0/C22:0 | 10% |
| Propylene Glycol | 5% |
| AMODIMETHICONE | 2% |
| Thioglycolic acid | qs for pH 1.5-2 |
| PANTHENOL | 0.50% |
| Fragrance | 0.25% |
| Demineralized water | qs for 100% |

Post-Dyeing Hair Treatment:

| Ingredients | % Material |
|---|---|
| Propylene Glycol | 10% |
| GBAC16:0/C22:0 | 8% |
| GBEC16:0 | 6% |
| AMODIMETHICONE | 2% |
| ISOPROPYL ALCOHOL | 2% |
| PANTHENOL | 0.50% |
| Fragrance | 0.25% |
| Colorant | 0.02% |
| Preservative | 0.01% |
| Demineralized water | qs for 100% |

Solid Conditioner:

| Ingredients | INCI | % Material |
|---|---|---|
| GBEC 18:0/C22:0 | ARACHIDYL/BEHENYL | 40 |
| | BETAINATE ESYLATE (AND) | |
| | ARACHIDYL/BEHENYL | |
| | ALCOHOL | |
| 1-Hexadecanol 98% | HEXADECANOL | 20 |
| Shea butter | BUTYROSPERMUM PARKII | 7 |
| | BUTTER | |
| Cocoa butter | THEOBROMA CACAO SEED | 3 |
| | BUTTER | |
| Argan oil | ARGANIA SPINOSA KERNEL | 3 |
| | OIL | |
| Refined hazelnut oil | CORYLUS AVELLANA SEED | 3 |
| | OIL | |
| Montanov 82 | ARACHIDYL ALCOHOL (AND) | 9.67 |
| | BEHENYL ALCOHOL (AND) | |
| | ARACHIDYL GLUCOSIDE | |
| L(+)-Sodium lactate | SODIUM LACTATE (AND) | 3.33 |
| 60% | AQUA | |
| Wheat starch | TRITICUM VULGARE STARCH | 8 |

Beard Balm:

| Ingredients | % Material |
|---|---|
| Shea butter | 50 |
| Castor oil | 15 |
| Sesame seed oil | 15 |
| Coconut oil | 10 |
| GBEC18:0/C22:0 | 9 |
| Tocopherol | 1 |

1-12. (canceled)

13. A cosmetic method for conditioning keratin fibers, comprising the topical application to the keratin fibers of a cosmetic composition in the form of an emulsion comprising, in a cosmetically acceptable medium, a surfactant composition comprising at least one glycine betaine derivative of formula (1): Xⁿ⁻[(CH₃)₃N⁺—CH₂—COZ—R]ₙ where Z denotes an oxygen atom or an —NH group, R is a saturated or unsaturated, linear or branched alkyl group comprising from 14 to 24 carbon atoms, X is an organic or inorganic anion, and n is equal to 1 or 2, provided that said surfactant composition does not contain alkyl polyglycosides.

14. The method according to claim 13, wherein the R radical is selected from the group consisting of: myristyl (C14:0), cetyl (C16:0), palmitoleyl (C16:1), stearyl (C18:0), oleyl (C18:1), linoleyl (C18:2), linolenyl (C18:3), arachidyl (C20:0), arachidonyl (C20:4), behenyl (C22:0), 2-hexyldecyl, 2-octyldodecyl and 2-decyltetradecyl.

15. The method according to claim 13, wherein the X anion is selected from the group consisting of: a chloride, a sulfate, a perchlorate, an alkylsulfate ion, decylsulfate, laurylsulfate, an arylsulfonate ion, an alkylsulfonate ion, and a sulfosuccinate ion.

16. The method according to claim 13, wherein the surfactant composition comprises the following constituents:
(a) at least one glycine betaine ester salt of formula (1): Xⁿ⁻[(CH₃)₃N⁺—CH₂—COO—R]ₙ where R is a saturated or unsaturated, linear or branched alkyl group comprising from 14 to 24 carbon atoms,
(b) at least one fatty alcohol of formula R—OH,
(c) an organic or inorganic acid of formula XH, and
(d) a glycine betaine salt of formula Xⁿ⁻[(CH₃)₃N⁺—CH₂—COOH]ₙ where X is an organic or inorganic anion and n is equal to 1 or 2.

17. The method according to claim 16, wherein the surfactant composition comprises:
(a) from 15% to 45% by weight of glycine betaine ester salt,
(b) from 55% to 80% by weight of fatty alcohol,
(c) from 0 to 5% by weight of organic or inorganic acid,
(d) from 0 to 3% by weight of glycine betaine salt,
(e) from 0 to 15% by weight of dialkyl ether.

18. The method according to claim 13, wherein the surfactant composition comprises the following constituents:
(a) one or more glycine betaine amide salts of formula (1): Xⁿ⁻[(CH₃)₃N⁺—CH₂—CONH—R]ₙ where R is a saturated or unsaturated, linear or branched alkyl group comprising from 14 to 24 carbon atoms;
(b) one or more alkylammonium salts of formula (2): Xⁿ⁻[NH₃⁺R]ₙ where R is a saturated or unsaturated, linear or branched alkyl group comprising from 14 to 24 carbon atoms;
(c) one or more glycine betaine ester salts of formula (3): Xⁿ⁻[(CH₃)₃ N⁺—CH₂—COOR′]ₙ where R′ is a saturated or unsaturated, linear or branched alkyl radical containing from 4 to 8 carbon atoms; and
(d) glycine betaine of formula (4): (CH₃)₃N⁺—CH₂—COO⁻ where X is an organic or inorganic anion and n is equal to 1 or 2.

19. The method according to claim 13, wherein the surfactant composition comprises at least 90% by weight of glycine betaine derivative.

20. The method according to claim 13, wherein the keratin fibers are selected from the group consisting of hair, beard and eyebrows.

21. The method according to claim 20, wherein the composition is applied to weakened and/or damaged hair or hair weakened and/or damaged by chemical treatments, mechanical treatments, dyeing, bleaching, permanent waving, straightening or by brushing.

22. The method according to claim 13, the conditioned keratin fibers having improved combability and/or softness and/or suppleness and/or manageability and/or sheen and/or smoothness and/or hydration and/or reduced static electricity.

23. The method according to claim 13, wherein the cosmetic composition is applied to hair that has been washed and rinsed beforehand.

24. The method according to claim 13, wherein the X anion is selected from the group consisting of alkylsulfonates and arylsulfonates.

25. The method according to claim 16, wherein R is a saturated or unsaturated, linear or branched alkyl group comprising from 18 to 22 carbon atoms.

26. The method according to claim 18, wherein R is a saturated or unsaturated, linear or branched alkyl group comprising from 16 to 22 carbon atoms.

27. The method according to claim 24, wherein the alkylsulfonate is methanesulfonate or ethanesulfonate.

28. A keratin fiber conditioning agent, comprising a surfactant composition comprising at least one glycine betaine derivative of formula (1): Xⁿ⁻[(CH₃)₃N⁺—CH₂—COZ—R]ₙ where Z denotes an oxygen atom or an —NH group, R is a saturated or unsaturated, linear or branched alkyl group comprising from 14 to 24 carbon atoms, X is an organic or inorganic anion, and n is equal to 1 or 2.
